(19) United States
(12) Patent Application Publication
(10) Pub. No.: US 2023/0404462 A1
(43) Pub. Date: Dec. 21, 2023

(54) METHOD FOR MANAGING HEALTH CONDITION OF GROWN-UP CATTLE, SYSTEM FOR MANAGING HEALTH CONDITION OF GROWN-UP CATTLE, APPARATUS FOR MANAGING HEALTH CONDITION OF GROWN-UP CATTLE, AND GARMENT FOR MANAGING HEALTH CONDITION OF GROWN-UP CATTLE

(71) Applicant: TOYOBO CO., LTD., Osaka (JP)

(72) Inventor: Yoko KOMATSU, Shiga (JP)

(73) Assignee: TOYOBO CO., LTD., Osaka (JP)

(21) Appl. No.: 18/037,355

(22) PCT Filed: Nov. 9, 2021
(86) PCT No.: PCT/JP2021/041177

§ 371 Date: May 17, 2023

(30) Foreign Application Priority Data
Nov. 20, 2020 (JP) 2020-193644

(51) Int. Cl. A61B 5/352; G16H 50/70; A61B 5/024; A61B 5/00; A01K 29/00

(52) U.S. Cl. CPC A61B 5/352; G16H 50/70; A61B 5/024; A61B 5/6804; A01K 29/005; A61B 2503/40; A01K 27/002

(57) ABSTRACT

An object of the present invention is to provide a method for easily managing a health condition of grown-up cattle. The method for managing a health condition of grown-up cattle, including: calculating at least one (hereinafter referred to as beat values) of average values of the beat intervals, variations in the beat intervals, beat numbers, HFs, or LF/HFs in the predetermined period; obtaining a regression formula of the beat values and the accelerations, and obtaining a beat value when an acceleration is 0 (hereinafter referred to as a time A₀) using the regression formula; and determining that the grown-up cattle is in a fasting state when a magnitude relationship of at least one of following formulae (1) to (5) is satisfied, and determining that the grown-up cattle is in a non-fasting state when none of magnitude relationships of following formulae (1) to (5) is satisfied:

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5).

### TECHNICAL FIELD

The present invention relates to a method for managing a health condition of grown-up cattle, a system for managing a health condition of grown-up cattle, an apparatus for managing a health condition of grown-up cattle, and a garment for managing a health condition of grown-up cattle.

### BACKGROUND ART

In recent years, apparatuses and systems for measuring biological information of animals have attracted attention. For example, Patent Document 1 discloses a delivery monitoring apparatus including a heart rate meter that measures a heart rate of an animal mother whose delivery is to be monitored, a circuit that discriminates a temporary decrease in the heart rate from the heart rate detected by the heart rate meter, the temporary decrease exceeding a fluctuation range of the heart rate in a normal time before delivery, and a communication apparatus and/or an alarm apparatus that notify a signal immediately before delivery due to the decrease in the heart rate output from the discrimination circuit.

### RELATED ART DOCUMENT

### Patent Document

Patent Document 1: JP-A-2008-011916

### SUMMARY OF THE INVENTION

### Problems to be Solved by the Invention

Grown-up cattle of dairy cattle, beef cattle, or the like instinctively tries to hide a poor physical condition even if the poor physical condition occurs due to, for example, food shortage, so that it is hard for an owner or the like to notice the poor physical condition of the cattle. In addition, even if the poor physical condition is mild, the continuous poor physical condition may cause deterioration in milk quality and meat quality, reduction in milk production, reduction in muscle amount, or the like. Therefore, in recent years, there has been a demand for development of a method or the like for easily managing a health condition of grown-up cattle. The present invention has been made in view of the above situations, and an object of the present invention is to provide a method for easily managing a health condition of grown-up cattle. Another object of the present invention is to provide a system, an apparatus, and a garment for easily managing a health condition of grown-up cattle.

### Solution to the Problems

A method for managing a health condition of grown-up cattle according to an embodiment of the present invention capable of solving the above problems is as described in [1].

[1] A method for managing a health condition of grown-up cattle, including:
measuring beat intervals and accelerations in a predetermined period;
calculating at least one (hereinafter referred to as beat values) of average values of the beat intervals, variations in the beat intervals, beat numbers, HFs, or LF/HFs in the predetermined period;
obtaining a regression formula of the beat values and the accelerations, and obtaining a beat value when an acceleration is 0 (hereinafter referred to as a time A₀) using the regression formula; and
determining that the grown-up cattle is in a fasting state when a magnitude relationship of at least one of following formulae (1) to (5) is satisfied, and determining that the grown-up cattle is in a non-fasting state when none of magnitude relationships of following formulae (1) to (5) is satisfied:

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)
in the formulae (1) to (5), A₁ to A₅ are constants,
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

As described above, when the magnitude relationship of at least one of formulae (1) to (5) relating to a beat value when an acceleration of the grown-up cattle is 0 is satisfied, it is possible to determine that the grown-up cattle is in the fasting state. Furthermore, it is possible to easily maintain the health condition of the grown-up cattle by the owner, an apparatus, or the like performing necessary treatment such as feeding on the basis of this determination result. A preferable aspect of the method for managing the health condition of the grown-up cattle is any one of [2] to [8], [1a] to [1d], and [3a] to [3d] below.

[2] The method for managing a health condition of grown-up cattle according to [1], including setting, as negative acceleration, a value obtained by multiplying acceleration in a body height direction at a time of standing of the grown-up cattle by −1 when the acceleration in the body height direction is a positive value, setting a negative value as the negative acceleration when the acceleration is the negative value, and determining that the grown-up cattle is in a sufficient side-lying state when a time during which the negative acceleration is a constant T or more (hereinafter, referred to as a side-lying time) is a constant L or more, and determining that the grown-up cattle is in an insufficient side-lying state when the side-lying time is less than the constant L.

[3] The method for managing a health condition of grown-up cattle according to [2], including creating a two-dimensional coordinate system with the beat value at the time A₀ and the side-lying time as axes, and fractionating the two-dimensional coordinate system into four regions below:
Region 1: a region in which the beat value at the time A₀ does not satisfy any of the magnitude relationships of the formulae (1) to (5) and the side-lying time is the constant L or more
Region 2: a region in which the beat value at the time A₀ does not satisfy any of the magnitude relationships of the formulae (1) to (5) and the side-lying time is less than the constant L
Region 3: a region in which the beat value at the time A₀ satisfies at least one of the magnitude relationships of the formulae (1) to (5) and the side-lying time is the constant L or more
Region 4: a region in which the beat value at the time A₀ satisfies at least one of the magnitude relationships of the formulae (1) to (5) and the side-lying time is less than the constant L.

[4] The method for managing a health condition of grown-up cattle according to any one of [1] to [3], wherein each of the average values of the beat intervals is an average value of RRIs each of which is an interval between an R wave and an R wave in an electrocardiographic signal.

[5] The method for managing a health condition of grown-up cattle according to any one of [1] to [4], wherein each of the beat numbers in the predetermined period is a heart rate or a pulse rate.

[6] The method for managing a health condition of grown-up cattle according to any one of [1] to [5], wherein each of the variations in the beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

[7] The method for managing a health condition of grown-up cattle according to any one of [1] to [6], wherein the beat intervals are measured using a biological information measuring garment.

[8] The method for managing a health condition of grown-up cattle according to [7], wherein
the biological information measuring garment includes:
a clothing fabric; and
an electrode provided on a skin-side surface side of the clothing fabric,
the clothing fabric includes a first band portion on one-end portion side in a body circumferential direction and a second band portion on an other-end portion side in the body circumferential direction, and
a front region ahead of a region from the first band portion to the second band portion of the clothing fabric has an area larger than a rear region behind the region from the first band portion to the second band portion of the clothing fabric.

[1a] The method for managing a health condition of grown-up cattle according to any one of [1] to [8], wherein
in the calculating, at least one of the average values of the beat intervals, the variations in the beat intervals, or the HFs in the predetermined period is calculated, and
in the determining, it is determined that the grown-up cattle is in the fasting state when the magnitude relationship of at least one of formulae (1), (2), or (4) is satisfied, and it is determined that the grown-up cattle is in the non-fasting state when none of magnitude relationships of formulae (1), (2), and (4) is satisfied.

[1b] The method for managing a health condition of grown-up cattle according to any one of [1] to [8], wherein
in the calculating, the average values of the beat intervals in the predetermined period are calculated, and
in the determining, it is determined that the grown-up cattle is in the fasting state when the magnitude relationship of the formula (1) is satisfied, and it is determined that the grown-up cattle is in the non-fasting state when the magnitude relationship of the formula (1) is not satisfied.

[1c] The method for managing a health condition of grown-up cattle according to any one of [1] to [8], wherein
in the calculating, the variations in the beat intervals in the predetermined period are calculated, and
in the determining, it is determined that the grown-up cattle is in the fasting state when the magnitude relationship of the formula (2) is satisfied, and it is determined that the grown-up cattle is in the non-fasting state when the magnitude relationship of the formula (2) is not satisfied.

[1d] The method for managing a health condition of grown-up cattle according to any one of [1] to [8], wherein in the calculating, the HFs in the predetermined period are calculated, and
in the determining, it is determined that the grown-up cattle is in the fasting state when the magnitude relationship of the formula (4) is satisfied, and it is determined that the grown-up cattle is in the non-fasting state when the magnitude relationship of the formula (4) is not satisfied.

[3a] The method for managing a health condition of grown-up cattle according to any one of [3] to [8] and [Ra] to [1d], in which the regions 1 to 4 are regions below:
Region 1: a region in which the beat value at the time A₀ does not satisfy the magnitude relationships of formulae (1), (2) and (4), and the side-lying time is the constant L or more
Region 2: a region in which the beat value at the time A₀ does not satisfy the magnitude relationships of formulae (1), (2), and (4), and the side-lying time is less than the constant L
Region 3: a region in which the beat value at the time A₀ satisfies the magnitude relationship of at least one of formulae (1), (2), or (4), and the side-lying time is the constant L or more
Region 4: a region in which the beat value at the time A₀ satisfies the magnitude relationship of at least one of formulae (1), (2), or (4), and the side-lying time is less than the constant L.
[3b] The method for managing a health condition of grown-up cattle according to any one of [3] to [8] and [1a] to [1d], in which the regions 1 to 4 are regions below:
Region 1: a region in which the beat value at the time A₀ does not satisfy the magnitude relationship of formula (1), and the side-lying time is the constant L or more
Region 2: a region in which the beat value at the time A₀ does not satisfy the magnitude relationship of formula (1), and the side-lying time is less than the constant L
Region 3: a region in which the beat value at the time A₀ satisfies the magnitude relationship of formula (1), and the side-lying time is the constant L or more
Region 4: a region in which the beat value at the time A₀ satisfies the magnitude relationship of formula (1), and the side-lying time is less than the constant L.

[3c] The method for managing a health condition of grown-up cattle according to any one of [3] to [8] and [1a] to [1d], in which the regions 1 to 4 are regions below:
Region 1: a region in which the beat value at the time A₀ does not satisfy the magnitude relationship of formula (2), and the side-lying time is the constant L or more
Region 2: a region in which the beat value at the time A₀ does not satisfy the magnitude relationship of formula (2), and the side-lying time is less than the constant L
Region 3: a region in which the beat value at the time A₀ satisfies the magnitude relationship of formula (2), and the side-lying time is the constant L or more
Region 4: a region in which the beat value at the time A₀ satisfies the magnitude relationship of formula (2), and the side-lying time is less than the constant L.

[3d] The method for managing a health condition of grown-up cattle according to any one of [3] to [8] and [1a] to [1d], in which the regions 1 to 4 are regions below:
Region 1: a region in which the beat value at the time A₀ does not satisfy the magnitude relationship of formula (4), and the side-lying time is the constant L or more
Region 2: a region in which the beat value at the time A₀ does not satisfy the magnitude relationship of formula (4), and the side-lying time is less than the constant L
Region 3: a region in which the beat value at the time A₀ satisfies the magnitude relationship of formula (4), and the side-lying time is the constant L or more
Region 4: a region in which the beat value at the time A₀ satisfies the magnitude relationship of formula (4), and the side-lying time is less than the constant L.

A system for managing a health condition of grown-up cattle according to an embodiment of the present invention that has been able to solve the above problems is as described in [9], and preferable aspects are as described in any one of [10] to [15], [9a] to [9d], and [11a] to [11d].

[9] A system for managing a health condition of grown-up cattle, including:
a measurement unit that measures beat intervals and accelerations in a predetermined period;
a calculation unit that calculates at least one (hereinafter referred to as beat values) of average values of the beat intervals, variations in the beat intervals, beat numbers, HFs, or LF/HFs in the predetermined period;
a processing unit that obtains a regression formula of the beat values and the accelerations, and obtains a beat value when an acceleration is 0 (hereinafter referred to as a time A₀) using the regression formula; and
a determination unit that determines that the grown-up cattle is in a fasting state when a magnitude relationship of at least one of following formulae (1) to (5) is satisfied, and determines that the grown-up cattle is in a non-fasting state when none of magnitude relationships of following formulae (1) to (5) is satisfied:

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)
in the formulae (1) to (5), A₁ to A₅ are constants, provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

[10] The system for managing a health condition of grown-up cattle according to [9],
wherein the calculation unit sets, as negative acceleration, a value obtained by multiplying acceleration in a body height direction at a time of standing of the grown-up cattle by −1 when the acceleration in the body height direction is a positive value, sets a negative value as the negative acceleration when the acceleration is the negative value, and calculates a time during which the negative acceleration is a constant T or more (hereinafter, referred to as a side-lying time), and
the determination unit determines that the grown-up cattle is in a sufficient side-lying state when the side-lying time is a constant L or more, and determines that the grown-up cattle is in an insufficient side-lying state when the side-lying time is less than the constant L.

[11] The system for managing a health condition of grown-up cattle according to [9] or [10], wherein the determination unit creates a two-dimensional coordinate system with the beat value at the time A₀ and the side-lying time as axes and fractionates the two-dimensional coordinate system into four regions below:
Region 1: a region in which the beat value at the time A₀ does not satisfy any of the magnitude relationships of the formulae (1) to (5) and the side-lying time is the constant L or more
Region 2: a region in which the beat value at the time A₀ does not satisfy any of the magnitude relationships of the formulae (1) to (5) and the side-lying time is less than the constant L
Region 3: a region in which the beat value at the time A₀ satisfies at least one of the magnitude relationships of the formulae (1) to (5) and the side-lying time is the constant L or more
Region 4: a region in which the beat value at the time A₀ satisfies at least one of the magnitude relationships of the formulae (1) to (5) and the side-lying time is less than the constant L.

[12] The system for managing a health condition of grown-up cattle according to any one of [9] to [11], wherein each of the average values of the beat intervals is an average value of RRIs each of which is an interval between an R wave and an R wave in an electrocardiographic signal.

[13] The system for managing a health condition of grown-up cattle according to any one of [9] to [12], wherein each of the beat numbers in the predetermined period is a heart rate or a pulse rate.

[14] The system for managing a health condition of grown-up cattle according to any one of [9] to [13], wherein each of the variations in the beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

[15] The system for managing a health condition of grown-up cattle according to any one of [9] to [14],
wherein the measurement unit is provided on a clothing fabric of a biological information measuring garment,
the clothing fabric includes a first band portion on a one-end portion side in a body circumferential direction and a second band portion on an other-end portion side in the body circumferential direction, and
a front region ahead of a region from the first band portion to the second band portion of the clothing fabric has an area larger than a rear region behind the region from the first band portion to the second band portion of the clothing fabric.

[9a] The system for managing a health condition of grown-up cattle according to any one of [9] to [15], wherein
the calculation unit calculates at least one of the average values of the beat intervals, the variations in the beat intervals, or the HFs in the predetermined period, and
the determination unit determines that the grown-up cattle is in the fasting state when the magnitude relationship of at least one of formulae (1), (2), or (4) is satisfied, and determines that the grown-up cattle is in the non-fasting state when none of magnitude relationships of formulae (1), (2), and (4) is satisfied.

[9b] The method for managing a health condition of grown-up cattle according to any one of [9] to [15], wherein
in the calculating, the average values of the beat intervals in the predetermined period are calculated, and
the determination unit determines that the grown-up cattle is in the fasting state when the magnitude relationship of formula (1) is satisfied, and determines that the grown-up cattle is in the non-fasting state when the magnitude relationship of formula (1) is not satisfied.

[9c] The system for managing a health condition of grown-up cattle according to any one of [9] to [15], wherein
in the calculating, the variations in the beat intervals in the predetermined period are calculated, and
the determination unit determines that the grown-up cattle is in the fasting state when the magnitude relationship of formula (2) is satisfied, and determines that the grown-up cattle is in the non-fasting state when the magnitude relationship of formula (2) is not satisfied.

[9d] The system for managing a health condition of grown-up cattle according to any one of [9] to [15], wherein
in the calculating, the HFs in the predetermined period are calculated, and
the determination unit determines that the grown-up cattle is in the fasting state when the magnitude relationship of formula (4) is satisfied, and determines that the grown-up cattle is in the non-fasting state when the magnitude relationship of the formula (4) is not satisfied.

[11a] The system for managing a health condition of grown-up cattle according to any one of [9] to [15] and [9a] to [9d], in which the regions 1 to 4 are regions below:
Region 1: a region in which the beat value at the time A₀ does not satisfy the magnitude relationships of formulae (1), (2) and (4), and the side-lying time is the constant L or more
Region 2: a region in which the beat value at the time A₀ does not satisfy the magnitude relationships of formulae (1), (2), and (4), and the side-lying time is less than the constant L
Region 3: a region in which the beat value at the time A₀ satisfies the magnitude relationship of at least one of formulae (1), (2), or (4), and the side-lying time is the constant L or more
Region 4: a region in which the beat value at the time A₀ satisfies the magnitude relationship of at least one of formulae (1), (2), or (4), and the side-lying time is less than the constant L.

[11b] The system for managing a health condition of grown-up cattle according to any one of [9] to [15] and [9a] to [9d], in which the regions 1 to 4 are regions below:
Region 1: a region in which the beat value at the time A₀ does not satisfy the magnitude relationship of formula (1), and the side-lying time is the constant L or more
Region 2: a region in which the beat value at the time A₀ does not satisfy the magnitude relationship of formula (1), and the side-lying time is less than the constant L
Region 3: a region in which the beat value at the time A₀ satisfies the magnitude relationship of formula (1), and the side-lying time is the constant L or more
Region 4: a region in which the beat value at the time A₀ satisfies the magnitude relationship of formula (1), and the side-lying time is less than the constant L.

[11c] The system for managing a health condition of grown-up cattle according to any one of [9] to [15] and [9a] to [9d], in which the regions 1 to 4 are regions below:
Region 1: a region in which the beat value at the time A₀ does not satisfy the magnitude relationship of formula (2), and the side-lying time is the constant L or more
Region 2: a region in which the beat value at the time A₀ does not satisfy the magnitude relationship of formula (2), and the side-lying time is less than the constant L
Region 3: a region in which the beat value at the time A₀ satisfies the magnitude relationship of formula (2), and the side-lying time is the constant L or more
Region 4: a region in which the beat value at the time A₀ satisfies the magnitude relationship of formula (2), and the side-lying time is less than the constant L.

[11d] The system for managing a health condition of grown-up cattle according to any one of [9] to [15] and [9a] to [9d], in which the regions 1 to 4 are regions below:
Region 1: a region in which the beat value at the time A₀ does not satisfy the magnitude relationship of formula (4), and the side-lying time is the constant L or more
Region 2: a region in which the beat value at the time A₀ does not satisfy the magnitude relationship of formula (4), and the side-lying time is less than the constant L
Region 3: a region in which the beat value at the time A₀ satisfies the magnitude relationship of formula (4), and the side-lying time is the constant L or more
Region 4: a region in which the beat value at the time A₀ satisfies the magnitude relationship of formula (4), and the side-lying time is less than the constant L.

A garment for managing a health condition of grown-up cattle and an apparatus for managing a health condition of grown-up cattle according to an embodiment of the present invention that have been able to solve the above problems is as described in [16] and [17] and preferable aspects are as described in any one of [17a] to [17d].

[16] A garment including at least a part of the system for managing a health condition of grown-up cattle according to any one of [9] to [15], [9a] to [9d], and [11a] to [11d].

[17] An apparatus for managing a health condition of grown-up cattle, including:
a measurement unit that measures beat intervals and accelerations in a predetermined period;
a calculation unit that calculates at least one (hereinafter referred to as beat values) of average values of the beat intervals, variations in the beat intervals, beat numbers, HFs, or LF/HFs in the predetermined period;
a processing unit that obtains a regression formula of the beat values and the accelerations, and obtains a beat value when an acceleration is 0 (hereinafter referred to as a time A₀) using the regression formula; and
a determination unit that determines that the grown-up cattle is in a fasting state when a magnitude relationship of at least one of following formulae (1) to (5) is satisfied, and determines that the grown-up cattle is in a non-fasting state when none of magnitude relationships of following formulae (1) to (5) is satisfied:

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)

in the formulae (1) to (5), A₁ to A₅ are constants,
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

[17a] The apparatus for managing a health condition of grown-up cattle according to [17], wherein
the calculation unit calculates at least one of the average values of the beat intervals, the variations in the beat intervals, or the HFs in the predetermined period is calculated, and
the determination unit determines that the grown-up cattle is in the fasting state when the magnitude relationship of at least one of formulae (1), (2), or (4) is satisfied, and determines that the grown-up cattle is in the non-fasting state when none of magnitude relationships of formulae (1), (2), and (4) is satisfied.

[17b] The apparatus for managing a health condition of grown-up cattle according to [17], wherein
the calculation unit calculates the average values of the beat intervals in the predetermined period, and
the determination unit determines that the grown-up cattle is in the fasting state when the magnitude relationship of formula (1) is satisfied, and determines that the grown-up cattle is in the non-fasting state when the magnitude relationship of formula (1) is not satisfied.

[17c] The apparatus for managing a health condition of grown-up cattle according to [17], wherein
the calculation unit calculates the variation in the beat intervals in the predetermined period, and
the determination unit determines that the grown-up cattle is in the fasting state when the magnitude relationship of formula (2) is satisfied, and determines that the grown-up cattle is in the non-fasting state when the magnitude relationship of formula (2) is not satisfied.

[17d] The apparatus for managing a health condition of grown-up cattle according to [17], wherein
the calculation unit calculates the HFs in the predetermined period, and
the determination unit determines that the grown-up cattle is in the fasting state when the magnitude relationship of formula (4) is satisfied, and determines that the grown-up cattle is in the non-fasting state when the magnitude relationship of formula (4) is not satisfied.

### Effects of the Invention

According to the present invention, with the above configuration, it is possible to provide a method for easily managing a health condition of grown-up cattle. In addition, it is possible to provide a system, an apparatus, and a garment for easily managing a health condition of grown-up cattle.

### BRIEF DESCRIPTION OF THE DRAWINGS

FIG. 1 is a diagram illustrating one example of an electrocardiographic waveform.

FIG. 2 is a diagram illustrating one example of a pulse wave.

FIG. 3 is an explanatory diagram of power spectrum integration.

FIG. 4 is an explanatory diagram of a regression formula of beat value and accelerations.

FIG. 5 is an explanatory diagram of fractionation in a two-dimensional coordinate system.

FIG. 6 is a plan view of a front side of one example of a biological information measuring garment.

FIG. 7 is a view illustrating a state in which the biological information measuring garment of FIG. 6 is worn on cattle.

FIG. 8 is a block diagram illustrating a configuration of a system for managing a health condition of grown-up cattle according to a first embodiment.

FIG. 9 is a block diagram illustrating a configuration of a system for managing a health condition of grown-up cattle according to a second embodiment.

### MODE FOR CARRYING OUT THE INVENTION

A method for managing a health condition of grown-up cattle according to an embodiment of the present invention includes: measuring beat intervals and accelerations in a predetermined period; calculating at least one (hereinafter referred to as beat values) of average values of the beat intervals, variations in the beat intervals, beat numbers, HFs, or LF/HFs in the predetermined period; obtaining a regression formula of the beat values and the accelerations, and obtaining a beat value when an acceleration is 0 (hereinafter referred to as a time A₀) using the regression formula; and determining that the grown-up cattle is in a fasting state when a magnitude relationship of at least one of formulae (1) to (5) below is satisfied, and determining that the grown-up cattle is in a non-fasting state when none of magnitude relationships of formulae (1) to (5) below is satisfied:

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)
in formulae (1) to (5). A₁ to A₅ are constants,
provided that, each LF is a value obtained by performing definite integration of a power spectrum from frequencies Lf1 to Lf2, the power spectrum being obtained by including performing frequency spectrum conversion on beat intervals, and each HF is a value obtained by performing definite integration of the power spectrum from frequencies Hf1 to Hf2, where Hf1>Lf1 and Hf2>Lf2 are satisfied.

According to examination of the present inventor, it has been found that when the magnitude relationship of at least one of formulae (1) to (5) relating to a beat value when an acceleration of the grown-up cattle is 0 is satisfied, it is possible to determine that the grown-up cattle is in the fasting state. Particularly, according to the examination of the present inventor, it has been found that the fluctuation of the beat value of the grown-up cattle is affected by the acceleration and a feed consumption amount, and it has been found that the fasting state of the grown-up cattle can be determined by removing the factor of the acceleration using the regression formula. In particular, in the case of grown-up cattle that can freely move in a stable hut and grown-up cattle that are bred by grazing, an amount of exercise is large, so that influence of the acceleration is large, and it is difficult to determine the fasting state. However, by removing the factor of the acceleration by the regression formula as described above, it is possible to determine the fasting state of the grown-up cattle bred in an environment where it can freely exercise. Furthermore, it is possible to easily maintain the health condition of the grown-up cattle by the owner, an apparatus, or the like performing necessary treatment such as feeding on the basis of this determination result. Each of the steps will be described in detail below.

<Measuring Beat Intervals and Accelerations>

As each of the beat intervals, an R-R interval that is an interval between an R wave and an R wave in an electrocardiographic signal, an interval between a pulse wave and a pulse wave, or the like may be used.

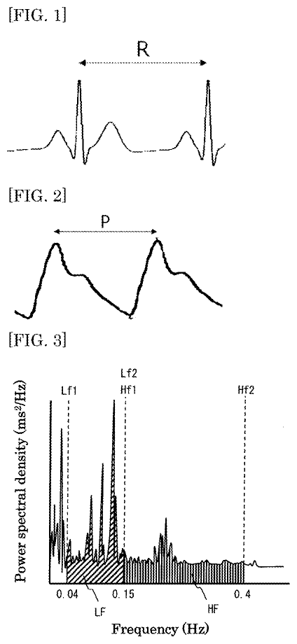

Among them, the R-R interval (RRI) is preferable. The RRI indicates an interval between an R wave and a next R wave of an electrocardiogram in which an electrical signal associated with beat of a heart is recorded in time series. Since a peak appears clearly in the R wave, erroneous recognition of a peak position hardly occurs, and measurement accuracy of the beat becomes high. Note that in FIG. 1 as one example of an electrocardiographic waveform, reference sign R corresponds to the RRI.

When a heartbeat signal is a pulse wave, it is preferable to use a P-P interval that is an interval between a P wave that is a pulse peak of a pulse wave, and a next P wave, the pulse wave being obtained by recording, in time series, a blood flow rate and a volume change of a blood vessel that change with pulsation of the heart. In the P-P interval, since the accuracy of the beat intervals increases because of clear appearance of the peak of the signal, erroneous recognition of the peak position hardly occurs. Note that in FIG. 2 as one example of the pulse wave, reference sign P corresponds to the P-P interval.

The beat intervals can be measured by an electrocardiograph that measures a heartbeat, a heartbeat sensor, or the like. These may have a beat interval measuring instrument such as electrodes or a stretchable capacitor. The electrodes are preferably fixed to a skin-side surface of clothing fabric of a garment. Examples of such stretchable electrodes include electrodes described in JP-A-2020-100903. Examples of the stretchable capacitor include a capacitor element described in JP-A-2019-072048. The electrocardiograph, the heartbeat sensor, and the like are preferably disposed on a chest and/or an abdomen of the grown-up cattle, and more preferably disposed on the chest.

The beat intervals may be measured using a pulse wave sensor. The pulse wave sensor, the electrocardiograph, the heartbeat sensor, and the like can be used in combination. In a case where a pulse is measured using the pulse wave sensor, near-infrared rays having a wavelength of 700 nm to 1200 nm are irradiated to an earlobe or the like of the grown-up cattle, and a reflection amount of the near-infrared rays can be measured in contact or non-contact.

The accelerations can be measured by an accelerometer or the like. Examples of the accelerometer include a uniaxial accelerometer, a biaxial accelerometer, and a triaxial accelerometer. Among them, the triaxial accelerometer is preferable. Examples of the accelerometer include myBeat manufactured by UNION TOOL Co. The accelerator or the like is preferably disposed on the chest and/or the abdomen of the grown-up cattle, and more preferably disposed on the chest.

The predetermined period for measuring the beat intervals and the accelerations is not particularly limited, and may be periods having different lengths. For example, the predetermined period for measuring the beat intervals is preferably 15 minutes or more, more preferably 30 minutes or more, still more preferably 1 hour or more, and preferably 12 hours or less, more preferably 8 hours or less, still more preferably 5 hours or less. The predetermined period for measuring the accelerations is preferably 5 hours or more, more preferably 12 hours or more, still more preferably 18 hours or more, and preferably 48 hours or less, more preferably 36 hours or less, still more preferably 28 hours or less. Furthermore, the predetermined period may be a sleep time zone, a time zone other than the sleep time zone, or a specific time zone such as 14:00 to 15:00.

<Calculating Beat Value>

In calculating the beat values, at least one of the average values of the beat intervals, the variations in the beat intervals, the beat numbers, the HFs, or the LF/HFs in the predetermined period is calculated on the basis of the measured beat intervals. It is preferable to calculate at least one of the average values of the beat intervals, the variations in the beat intervals, the HFs, or the LF/HFs in the predetermined period. It is more preferable to calculate at least one of the average values of the beat intervals, the variations in the beat intervals, or the HFs in the predetermined period. It is still more preferable to calculate at least one of the average values of the beat intervals, the variations in the beat intervals, or the HFs in the predetermined period.

Examples of the average value of the beat intervals include an average value of the R-R intervals each of which is an interval between an R wave and an R wave in the electrocardiographic signal, and an average value of the P-P intervals between the pulse wave and the pulse wave. Among them, the average value of the R-R intervals (RRIs) is preferable.

The variation in the beat intervals can be obtained by time-analyzing information relating to a time interval such as the RRI. Specifically, the variation in the beat intervals is preferably at least one of SDNN, RMSSD, CVRR, NN50, or pNN50. Provided that, the SDNN is a standard deviation of an RRI that is an interval between an R wave and an R wave in an electrocardiographic signal, the RMSSD is a square root of an average value of squares of differences between consecutive adjacent RRIs, the CVRR is a value obtained by dividing a value of the SDNN by an average value of RRIs and multiplying the obtained value by 100, the NN50 is a value indicating a total number of heartbeats in which the differences between the consecutive adjacent RRIs exceed 50 ms, and the pNN50 is a value indicating a ratio of the heartbeats in which the differences between the consecutive adjacent RRIs exceeds 50 ms. Among them, the SDNN or the RMSSD is preferable, and the RMSSD is more preferable.

The beat number in the predetermined period is preferably a heart rate or a pulse rate. Among them, the beat number is more preferably the beat number of heart beats. The beat number of heart beats can be calculated from the RRI, and can be obtained by, for example, a formula of 60/(RRI×0.001).

The HF and the LF/HF are usually used as indices of parasympathetic nerve activity and sympathetic nerve activity, but can be used as indices of the fasting state of the grown-up cattle. The HF and the LF can be each obtained, for example, by calculating a power spectrum of the RRI, the spectrum obtained by including performing frequency analysis, and integrating a power in a predetermined frequency region. For example, the LF can be obtained by definite integration from the frequencies Lf1 to Lf2, and the HF can be obtained by definite integration of the power spectrum from the frequencies Hf1 to Hf2. Here, Hf1>Lf1 and Hf2>Lf2 hold.

A detailed method for calculating the LF and the HF will be described with reference to FIG. 3 which is an explanatory diagram of power spectrum integration. In FIG. 3, a vertical axis represents a power spectral density (unit: ms²/Hz), and a horizontal axis represents a frequency (unit: Hz). The LF is a value obtained by performing definite integration of a power spectrum F², for example, from 0.04 Hz (Lf1) to 0.15 Hz (Lf2), and is an area of a portion hatched by oblique lines in FIG. 3. On the other hand, the HF is a value obtained by performing definite integration of the power spectrum F², for example, from 0.15 Hz (Hf1) to 0.40 Hz (Hf2), and is an area of a portion hatched by vertical lines in FIG. 3. In FIG. 3, an integration range is set such that both Lf2 and Hf1 are equal to 0.15 Hz, but is not limited to the above range. Relationships of Lf1<Hf1 and Lf2<Hf2 are preferably satisfied, and a relationship of Hf1>Lf2 is more preferably satisfied. In addition, the integration range of the LF may be 0.0133 to 0.20 Hz and the integration range of the HF may be 0.20 to 0.58 Hz.

It is preferable that the integration range of the LF includes at least 0.10 Hz and Lf1<0.10<Lf2 holds. Further, Lf1 is more preferably 0.01 to 0.05 Hz. Further, Lf2 is more preferably 0.10 to 0.30 Hz. It is preferable that the integration range of the HF includes at least 0.35 Hz and Hf1<0.35<Hf2 holds. Hf1 is more preferably 0.10 to 0.30 Hz. Hf2 is more preferably 0.38 to 0.60 Hz. In calculating the LF and the HF described above, for example, WO 2016/031650 A and the like can be referred to.

In calculating the beat value, it is not always necessary to calculate all the beat values, and at least one beat value may be calculated.

The beat value in the predetermined period is preferably calculated by an analysis apparatus of a system for managing a health condition of grown-up cattle described later or a calculation unit of a web server.

<Obtaining Beat Value when Acceleration is 0 (at Time of A₀)>

In obtaining a beat value when an acceleration is 0 (at the time A₀), the regression formula of the beat values and the accelerations is obtained, and the beat value at the time A₀ is obtained using the regression formula. For example, as illustrated in a scatter diagram of FIG. 4, the regression formula can be obtained by plotting a measurement result with the acceleration used as an explanatory variable x and the beat value such as the RRI used as an objective variable y and performing linear regression. In the regression analysis, a least square method may be performed. The beat value at the time A₀ can be obtained by obtaining the regression formula and applying x=0 to the regression formula for calculation.

In obtaining the regression formula, for example, the average value of the RRIs or the like per predetermined time in the predetermined period may be used as the beat value of the objective variable x, and an average value of acceleration per predetermined time in the predetermined period may be used as the acceleration of the objective variable y for plotting. The predetermined time may be 5 seconds or more, 10 seconds or more, 30 seconds or more, or 60 seconds or more, and may be 5 minutes or less, 4 minutes or less, 3 minutes or less, or 2 minutes or less.

Acceleration in a body height direction at the time of standing of the grown-up cattle, acceleration associated with movement, and the like may be used as the explanatory variable x. Among them, the acceleration associated with the movement of the grown-up cattle is preferable. The acceleration relating to the explanatory variable x may be calculated before the regression formula is obtained. In addition, as the acceleration in the body height direction at the time of standing, negative acceleration in the body height direction at the time of standing of the grown-up cattle described later may be used.

The acceleration associated with the movement (hereinafter, the acceleration may be referred to as acceleration A) is preferably a synthetic value of the acceleration of the grown-up cattle and a gravitational acceleration acting on the grown-up cattle, and the synthetic value may be represented by a ratio to a gravitational acceleration g (=9.8 m/s²) (unit: a dimensionless amount). Specifically, as expressed by a formula (I) below, the acceleration A is more preferably a value obtained by subtracting (g/g)=1 from a square root of a sum of squares of the acceleration x, y, and z in X-axis, Y-axis, and Z-axis directions, which are acceleration measured using a triaxial accelerometer, in which (g/g)=1 is the gravitational acceleration g (=9.8 m/s²) acting on the grown-up cattle (here, the unit g represents a magnitude of the gravitational acceleration). The acceleration A when there is no movement is 0.

[Mathematical formula 1]

Acceleration A=\sqrt{x²+y²+z²}−1 (I)
(wherein x, y, and z are X-axis, Y-axis, and Z-axis acceleration obtained from the accelerometer attached to the body of the grown-up cattle.)

The acceleration A may be a square root of a sum of squares of the acceleration x, y on the X-axis and the Y-axis, which are accelerations of the grown-up cattle measured using a biaxial accelerometer, and may be further represented as a ratio to the gravitational acceleration g (=9.8 m/s²) (unit: the dimensionless amount). In this case, the biaxial accelerometer is preferably attached to the grown-up cattle such that a measurement direction is perpendicular to a gravity direction.

The regression formula is preferably obtained for each of the grown-up cows. In addition, in order to estimate a state of a group of a plurality of grown-up cows, a certain grown-up cow may be used for a representative value.

The acceleration relating to the explanatory variable x, and the beat value may be calculated by an accelerometer or the like, or may be calculated by the analysis apparatus of the system for managing a health condition of grown-up cattle described later, the calculation unit of the web server, or the like. In addition, the regression formula and the beat value at the time A₀ may be calculated by the analysis apparatus of the system for managing a health condition of grown-up cattle described later, the processing unit of the web server, or the like.

<Determining Fasting State>

In determining the fasting state, it is determined that the grown-up cattle is in the fasting state when the magnitude relationship of at least one of above formulae (1) to (5) is satisfied, and it is determined that the grown-up cattle is in the non-fasting state when none of magnitude relationships of above formulae (1) to (5) is satisfied.

When the average value of the beat intervals is the RRI (ms), the constant A₁ is preferably 800 ms, more preferably 1000 ms, and still more preferably 1200 ms.

When the variation in the beat intervals is the SDNN, the constant A₂ is preferably 40, more preferably 140, and still more preferably 240.

When the variation in the beat intervals is the RMSSD, the constant A₂ is preferably 12, more preferably 120, and still more preferably 240. When the variation in the beat intervals is the CVRR, the constant A₂ is preferably 25, more preferably 35, and still more preferably 45. When the variation in the beat intervals is the NN50, the constant A₂ is preferably 30, more preferably 45, and still more preferably 60. When the variation in the beat intervals is the pNN50, the constant A₂ is preferably 80, more preferably 90, and still more preferably 100.

When the beat number is the heat rate, the constant A₃ is preferably 75, more preferably 60, and still more preferably 50.

The constant A₄ relating to the HF is preferably 1000, more preferably 3000, and still more preferably 5000.

The constant A₅ relating to the LH/HF is preferably 75, more preferably 50, and still more preferably 25.

In determining the fasting state, it is determined that the grown-up cattle is in the fasting state when the magnitude relationship of at least one of above formulae (1) to (5) is satisfied. Among them, it is preferable to determine that the grown-up cattle is in the fasting state when the magnitude relationship of at least one of above formulae (1), (2), (4), or (5) is satisfied, it is more preferable to determine that the grown-up cattle is in the fasting state when the magnitude relationship of at least one of above formulae (1), (2), or (4) is satisfied, and it is still more preferable to determine that the grown-up cattle is in the fasting state when the magnitude relationship of above formula (1), (2), or (4) is satisfied.

The method for managing a health condition of grown-up cattle may further include notifying information on the determination result of the fasting state. The information of the determination result is preferably perceptible information, and examples thereof include sound, vibration, image, and the like. The information may be notified using a perceptual information generator or the like such as a sound generator, a vibration generator, or a display. Examples of the sound generator include a sound generator that emits a warning sound for notifying the fasting state. Examples of the vibration generator include a vibration generator that generates vibration for notifying the fasting state. Examples of the display include a display that displays character information such as the fasting state and necessity of feeding; drawing information in which a region A indicating the fasting state and a region B indicating the non-fasting state are illustrated and the corresponding region is plotted, colored, blinked, or the like; composite information of the foregoing, and the like. In this step, it is preferable that information on the determination result is notified from a determination result notifying unit of the system for managing a health condition of grown-up cattle described later.

When the determination result is the fasting state, treatment such as increasing an amount of feed given or adjusting a blending ratio of the feed may be performed. In addition, when the determination result is the fasting state, a treatment signal may be generated by a determination unit of the system for managing a health condition of grown-up cattle described later. The treatment signal is transmitted to, for example, an automatic feeder or the like, and the grown-up cattle may be automatically fed on the basis of the treatment signal.

It is preferable that the method for managing a health condition of grown-up cattle includes setting, as negative acceleration, a value obtained by multiplying acceleration in a body height direction at a time of standing of the grown-up cattle by −1 when the acceleration in the body height direction is a positive value, setting a negative value as the negative acceleration when the acceleration is the negative value, and determining that the grown-up cattle is in a sufficient side-lying state when a time during which the negative acceleration is a constant T or more (hereinafter, referred to as a side-lying time) is a constant L or more, and determining that the grown-up cattle is in an insufficient side-lying state when the side-lying time is less than the constant L.

It can be said that the body height direction corresponds to a vertical direction when the grown-up cattle stands, and corresponds to a direction from a breast to a back of the grown-up cattle. For example, when, for example, the triaxial accelerometer is attached to the chest, the acceleration in the body height direction is measured as approximately 1 G or −1 G due to an influence of the gravitational acceleration when the grown-up cattle is in a standing position and is at rest, and the acceleration in the body height direction is measured as approximately 1 G or −1 G even when the grown-up cattle is in a prone position and is at rest. However, when the grown-up cattle is side-lying, the body height direction and the gravity direction approach to be perpendicular to each other, and thus the acceleration in the body height direction becomes close to 0 G. Therefore, the acceleration in the body height direction is effective for determining a side-lying position of the grown-up cattle. Furthermore, the acceleration in the body height direction is switched between positive and negative depending on an attachment direction of the accelerometer. However, by calculating the negative acceleration as described above, the side-lying time can be specified on the basis of the constant T regardless of the attachment direction.

If the determination result is a state in which the grown-up cattle are not at a side-lying position or the side-lying time is insufficient (i.e., the insufficient side-lying state), the owner or the like may perform treatment for adjusting a breeding environment such as adjusting environmental temperature and humidity, blowing air, replacing litter, or improving equipment of a stable. This makes it easy for the grown-up cattle to lie on its side. By sufficiently securing, particularly, the side-lying time in a lying position of the grown-up cattle, a blood flow rate to the breast of the grown-up cattle increases, and an increase in milk production can be expected. In particular, by sufficiently securing the side-lying time, a burden weight on the legs and the hoof of the adult cow is alleviated and the hoof disease can be prevented, and further, an increase in milk production, an increase in weight, and the like can be expected by an increase in the rumination and chewing time of the grown-up cattle.

When the grown-up cattle is in the above insufficient side-lying state, the treatment signal may be generated from the determination unit of the system for managing a health condition of grown-up cattle described later. The treatment signal may be transmitted to, for example, a predetermined apparatus, and the apparatus may perform necessary treatment on the basis of the treatment signal. Examples of the predetermined apparatus include a heater such as a fan heater, a far-infrared heater, or an oil heater, heating equipment such as an air conditioner, an electric fan, an air conditioner, a water supply machine, a washing machine, a belt conveyer for discharging feces and urine, and the like.

The constant T relating to the negative acceleration is preferably −0.7, more preferably −0.6, and still more preferably −0.5.

The constant L relating to the side-lying time may be, for example, any of 10 hours, 11 hours, 12 hours, 13 hours, and 14 hours, and is preferably 11 hours, 12 hours, or 13 hours, and more preferably 12 hours.

In determining that the grown-up cattle is in the sufficient side-lying state when the time during which the negative acceleration is the constant T or more (the side-lying time) is the constant L or more, it is preferable to determine the sufficient side-lying state in units of one day (24 hours). For example, it is preferable that the acceleration in the body height direction at the time of standing of the grown-up cattle is measured for 24 hours or more, and the sufficient side-lying state is determined when the time (side-lying time) during which the negative acceleration is the constant T or more is the constant L per 24 hours (e.g., 12 hours) or more.

The method for managing a health condition of grown-up cattle preferably includes creating a two-dimensional coordinate system with the beat value at the time A₀ and the side-lying time as axes and fractionating the two-dimensional coordinate system into four regions below:
Region 1: a region in which the beat value at the time A₀ does not satisfy any of the magnitude relationships of formulae (1) to (5) and the side-lying time is the constant L or more
Region 2: a region in which the beat value at the time A₀ does not satisfy any of the magnitude relationships of formulae (1) to (5) and the side-lying time is less than the constant L
Region 3: a region in which the beat value at the time A₀ satisfies at least one of the magnitude relationships of formulae (1) to (5) and the side-lying time is the constant L or more
Region 4: a region in which the beat value at the time A₀ satisfies at least one of the magnitude relationships of formulae (1) to (5) and the side-lying time is less than the constant L.

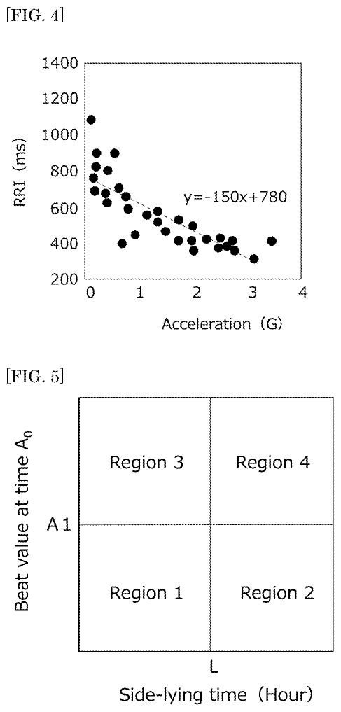

FIG. 5 is a two-dimensional coordinate system in which the beat value at the time A₀ is on a vertical axis and the side-lying time is on a horizontal axis, and illustrates one example of the fractionation into the four regions. By plotting the beat value at the time A₀ and the side-lying time of the grown-up cattle to be managed, and determining to which of the regions 1 to 4 the beat value and the side-lying time correspond, it is possible to objectively grasp a cause of a poor physical condition of the grown-up cattle. For example, in a case corresponding to the region 1, the grown-up cattle is in the non-fasting state, the side-lying time is sufficient, and thus the grown-up cattle is in a heathy condition. In a case corresponding to the region 2, while the grown-up cattle is in the non-fasting state, the side-lying time is insufficient, and thus a cause of the poor physical condition is considered to be an insufficient side-lying time. In a case corresponding to the region 3, while the grown-up cattle is in the fasting state, the side-lying time is sufficient, and thus a cause of the poor physical condition is considered to be a food shortage. In a case corresponding to the region 4, while the grown-up cattle is in the fasting state, the side-lying time is insufficient, and thus causes of the poor physical condition are considered to be a food shortage and an insufficient side-lying time.

The two-dimensional coordinate system divided into the four regions is preferably displayed by a display or the like. In the display or the like, the corresponding region is preferably plotted, colored, blinked, or the like on the basis of the measurement result. As a result, what state the health condition of the grown-up cattle can be easily grasped at a glance, and the owner, the apparatus, and the like can perform necessary treatment. By such a method for managing a health condition, it is possible to improve milk production, milk quality, and meat quality of the grown-up cattle. In addition, management of pregnancy, delivery, and the like may also be performed by the method for managing the health condition.

The grown-up cattle is preferably grown-up cattle having an age of 9 months or more. The age in month is more preferably 12 months or more, still more preferably 17 months or more, and still more preferably 24 months or more.

In the method for managing a health condition of grown-up cattle, the beat intervals are preferably measured using a biological information measuring garment. The garment preferably has a beat intervals measuring instrument that measures the beat intervals such as an electrocardiograph. A form of the garment is not particularly limited, and a belt-shaped article such as underwear or a belt is preferable. In addition, the garment preferably covers at least one of the chest, the abdomen, a leg, a neck, and a head, more preferably covers the chest and/or the abdomen, and still more preferably covers the chest. In addition, the garment preferably includes a detachable electronic unit.

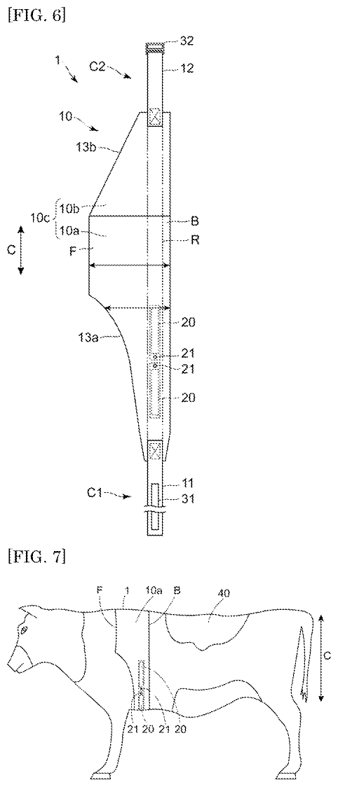

Examples of the biological information measuring garment include a biological information measuring garment 1 illustrated in FIG. 6. As illustrated in FIG. 6, it is preferable that the biological information measuring garment 1 includes clothing fabric 10 and electrodes 20 provided on a skin-side surface side of the clothing fabric 10, the clothing fabric 10 includes a first band portion 11 on a one-end portion C1 side in a body circumferential direction C and a second band portion 12 on an other-end portion C2 side in the body circumferential direction C, and a front region F ahead of a region (central region R) from the first band portion 11 to the second band portion 12 of the clothing fabric 10 has an area larger than a rear region B behind the central region R of the clothing fabric 10. Since the area of the front region F of the clothing fabric 10 is larger than the area of the rear region B on the rear side of the clothing fabric 10, for example, when the biological information measuring garment 1 is worn on grown-up cattle 40 as illustrated in FIG. 7, a positional deviation of the electrodes 20 hardly occurs, so that a measurement accuracy of the biological information is improved. Note that in FIG. 7, the front side means a head side of the grown-up cattle 40, and the rear side means a tail side of the grown-up cattle 40.

The first band portion 11 and the second band portion 12 may be fixed to each other directly or indirectly when the biological information measuring garment 1 is worn on the grown-up cattle 40. For example, as illustrated in FIG. 6, each of the first band portion 11 and the second band portion 12 preferably has a first coupling member 31 and a second coupling member 32. Examples of the first coupling member 31 and the second coupling member 32 include hook-and-loop fasteners such as Magic Tape (registered trademark) and Free Magic Tape (registered trademark), buckles, and the like. The first coupling member 31 and the second coupling member 32 may be coupled via, for example, a detachable belt. This can make it easy to adjust a length in the body circumferential direction C, so that the biological information measuring garment 1 can be easily worn.

In FIG. 6, the clothing fabric 10 has a main body portion 10c, the first band portion 11, and the second band portion 12, and the main body portion 10c is longer in a front-rear direction than the first band portion 11 and the second band portion 12. For example, as illustrated in FIG. 6, the main body portion 10c may have a first tapered portion 13a whose length in the front-rear direction decreases toward the first band portion 11. In addition, as illustrated in FIG. 6, the main body portion 10c may have a second tapered portion 13b whose length in the front-rear direction decreases toward the second band portion 12. This can make it easy to tighten the biological information measuring garment 1. Examples of shapes of the first tapered portion 13a and the second tapered portion 13b include a curved shape in which an outer edge is curved toward a rear direction, a so-called linear tapered shape in which the outer edge is tapered off at a constant angle, and the like. Note that the main body portion 10c may have a rectangular shape such as a square shape or an oblong shape.

Note that the electrodes 20 in FIG. 6 are formed by providing a first insulating layer on a skin-side surface of the clothing fabric 10 and providing a conductive layer on the first insulating layer. Further, a second insulating layer is provided on the conductive layer other than the electrodes 20 and electronic equipment connection portions 21. In each of the electronic equipment connection portions 21, for example, electronic equipment such as the accelerometer or the electrocardiograph can be attached to a front surface of the clothing fabric 10 via a connector such as a snap fastener.

These methods for managing a health condition of grown-up cattle can be implemented using, for example, the system for managing a health condition of grown-up cattle described in detail below or an apparatus for managing a health condition of grown-up cattle. At least some of the steps of the method for managing a health condition of grown-up cattle may be performed by the owner or the like of the grown-up cattle, and all the steps may be performed by the system or the apparatus for managing a health condition of grown-up cattle.

A system for managing a health condition of grown-up cattle according to an embodiment includes a measurement unit that measures beat intervals and accelerations in a predetermined period; a calculation unit that calculates at least one (hereinafter referred to as beat values) of average values of the beat intervals, variations in the beat intervals, beat numbers, HFs, or LF/HFs in the predetermined period; a processing unit that obtains a regression formula of the beat values and the accelerations, and obtains a beat value when an acceleration is 0 (hereinafter referred to as a time A₀) using the regression formula; and a determination unit that determines that the grown-up cattle is in a fasting state when a magnitude relationship of at least one of formulae (1) to (5) below is satisfied, and determining that the grown-up cattle is in a non-fasting state when none of magnitude relationships of formulae (1) to (5) below is satisfied.

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)
in formulae (1) to (5), A₁ to A₅ are constants, provided that, each LF is a value obtained by performing definite integration of a power spectrum from frequencies Lf1 to Lf2, the power spectrum being obtained by including performing frequency spectrum conversion on beat intervals, and each H F is a value obtained by performing definite integration of the power spectrum from frequencies Hf1 to Hf2, where Hf1>Lf1 and Hf2>Lf2 are satisfied.

As described above, the system for managing a health condition of grown-up cattle determines that the grown-up cattle is in the fasting state when the magnitude relationship of at least one of formulae (1) to (5) relating to a beat value when an acceleration of the grown-up cattle is 0 is satisfied as described above. By using the system, it is possible to create an index of the fasting state and notify the owner, the apparatus, and the like of the fasting state, and the owner, the apparatus, and the like can perform necessary treatment. For the beat intervals, the accelerations, the beat values, the beat value at the time A₀, formulae (1) to (5), and the like, the description of the method for managing a health condition of grown-up cattle can be referred to.

It is preferable that the calculation unit sets, as negative acceleration, a value obtained by multiplying the acceleration in the body height direction at a time of standing of the grown-up cattle by −1 when the acceleration in the body height direction is a positive value, sets a negative value as the negative acceleration when the acceleration is the negative value, and calculates a time when the negative acceleration is a constant T or more (hereinafter, referred to as a side-lying time).

It is preferable that the determination unit determines that the state is a sufficient side-lying state when the side-lying time is a constant L or more, and determines that the state is an insufficient side-lying state when the side-lying time is less than the constant L.

For the negative acceleration, the side-lying time, the sufficient side-lying state, the insufficient side-lying state, and the like, the description of the method for managing a health condition of grown-up cattle can be referred to.

It is preferable that the determination unit creates a two-dimensional coordinate system with the beat value at the time A₀ and the side-lying time as axes and fractionates the two-dimensional coordinate system into four regions below:
Region 1: a region in which the beat value at the time A₀ does not satisfy any of the magnitude relationships of formulae (1) to (5) and the side-lying time is the constant L or more
Region 2: a region in which the beat value at the time A₀ does not satisfy any of the magnitude relationships of formulae (1) to (5) and the side-lying time is less than the constant L
Region 3: a region in which the beat value at the time A₀ satisfies at least one of the magnitude relationships of formulae (1) to (5) and the side-lying time is the constant L or more
Region 4: a region in which the beat value at the time A₀ satisfies at least one of the magnitude relationships of formulae (1) to (5) and the side-lying time is less than the constant L.

For the beat intervals, the accelerations, the beat values, the beat value at the time A₀, formulae (1) to (5), the side-lying time, the two-dimensional coordinate system, and the like, the description of the method for managing a health condition of grown-up cattle can be referred to.

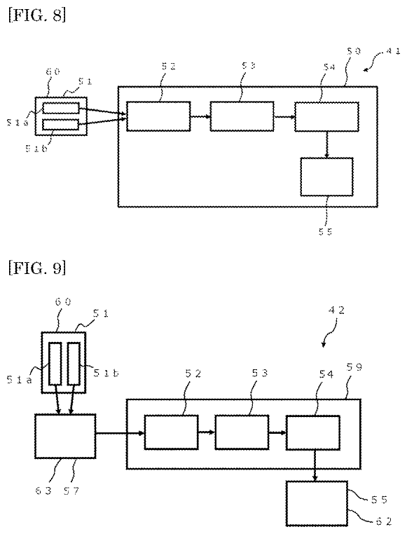

Hereinafter, a configuration and a function of a system for managing a health condition of grown-up cattle 41 according to a first embodiment will be described with reference to FIG. 8. Description of the matters described in the method for managing a health condition of grown-up cattle will be omitted.

As illustrated in FIG. 8, the system for managing a health condition of grown-up cattle 41 has a measurement instrument 60 and an analysis apparatus 50. In addition, the analysis apparatus 50 has a measurement unit 51, a calculation unit 52, a processing unit 53, a determination unit 54, and a determination result notifying unit 55. Further, the measurement instrument 60 may have a beat interval measuring unit 51a and an acceleration measuring unit 51b. One example of a flow of the system for managing a health condition of grown-up cattle 41 will be described below.

The measurement instrument 60 is attached to the chest or the like of the grown-up cattle, and the beat interval measuring unit 51a and the acceleration measuring unit 51b of the measurement unit 51 measure the beat intervals and the accelerations. Subsequently, the measurement result is transmitted to the calculation unit 52, and the calculation unit 52 calculates the negative accelerations in the body height direction of the grown-up cattle, and the beat values. The negative accelerations and the beat values may be calculated by the measurement unit 51. Subsequently, the processing unit 53 obtains the regression formula of the negative accelerations in the body height direction and the beat values, and further obtains a beat value when a negative acceleration in the body height direction is 0 (at the time A₀) using the regression formula. Furthermore, these calculation results are sent to the determination unit 54, and the determination unit 54 determines that the grown-up cattle is in the fasting state when the magnitude relationship of at least one of above formulae (1) to (5) is satisfied. These determination results are sent to the determination result notifying unit 55, and are notified in a perceivable form of information indicating that the grown-up cattle is in the fasting state. The determination result notifying unit 55 performs, for example, display indicating that the grown-up cattle is in the fasting state, generation of a warning sound for notifying that the grown-up cattle is in the fasting state, and the like.

Note that at least part of data obtained by the calculation unit 52 and the processing unit 53 may be directly transmitted to the determination result notifying unit 55.

It is preferable that the determination unit 54 calculates the side-lying time, determines that the grown-up cattle is in the sufficient side-lying state, creates the two-dimensional coordinate system, and fractionates the two-dimensional coordinate system, as described above. One example of a flow in that case will be described below.

First, similarly to the above flow, the beat intervals and the accelerations are measured, the measurement result is transmitted to the calculation unit 52, and the calculation unit 52 calculates the negative accelerations in the body height direction of the grown-up cattle and the beat values. Furthermore, the calculation unit 52 calculates the side-lying time on the basis of the negative accelerations in the body height direction. The accelerations, the beat values, and the side-lying time may be calculated by the measurement unit 51. Subsequently, the processing unit 53 obtains the regression formula of the negative accelerations in the body height direction and the beat values, and further obtains a beat value when a negative acceleration in the body height direction is 0 (at the time A₀) using the regression formula. These calculation results are sent to the determination unit 54, and the determination unit 54 creates the two-dimensional coordinate system with the beat value at the time A₀ and the side-lying time as axes, fractionates the two-dimensional coordinate system into the four regions, and determines which one of the four regions the calculation results correspond to. The determination result is sent to the determination result notifying unit 55, and is notified in the perceivable form of information. The determination result notifying unit 55 performs, for example, the display, the generation of the warning sound, or the like indicating that the grown-up cattle is in the fasting state or in the insufficient side-lying state.

Examples of the measurement instrument 60 include an electrocardiograph that measures a heartbeat of the grown-up cattle, a heartbeat sensor, a pulse wave sensor that measures a pulse, an acceleration sensor, and the like. These can be used singly or in combination of two or more kinds thereof. The measurement unit 51 may measure the beat intervals such as the RRIs, and the accelerations. The beat interval measuring unit 51a and the acceleration measuring unit 51b do not need to be configured by the same measurement instrument, and may be configured by different measurement instruments. The measurement instrument 60 may be formed integrally with the analysis apparatus 50.

As illustrated in FIG. 8, the calculation unit 52, the processing unit 53, the determination unit 54, and the determination result notifying unit 55 may be provided as configuration units of the analysis apparatus 50, and at least some of them may be provided as configuration units of the measurement instrument 60. The units may be configured of a processor such as a central processing unit (CPU) or a microprocessor (MPU), a storage medium such as a random access memory (RAM) or a read only memory (ROM), a logic circuit such as a field programmable gate array (FPGA), a large scale integration (LSI), or an application specific integrated circuit (ASIC), some or all of them, and the like. In addition, the analysis apparatus 50 may have an auxiliary storage device such as a hard disk drive (HDD) or a flash memory, and a portable recording medium such as a CD-ROM, a DV) disk, or a USB memory.

The information notified from the determination result notifying unit 55 may be perceptible information or a signal for another apparatus (not illustrated) to perform necessary processing. Examples of the perceptible information include sound, vibration, and image. Examples of a determination result notifier configuring the determination result notifying unit 55 include a sound generator, a vibration generator, a display, a signal generator, and the like. These may be used singly or in combination of two or more kinds thereof. In addition, the determination result notifier may be provided inside the analysis apparatus 50 or may be provided outside the analysis apparatus 50.

Examples of the above other apparatus include an automatic feeder, a heater such as a fan heater, a far-infrared heater, or an oil heater, heating equipment such as an air conditioner, an electric fan, an air conditioner, a water supply machine, a washing machine, a belt conveyer for discharging feces and urine, and the like.

The transmission of the signal from the measurement unit 51 to the calculation unit 52 may be wired or wireless. In addition, the analysis apparatus 50 may have a reception unit (not illustrated) that receives the signal from the measurement unit 51. Furthermore, the analysis apparatus 50 may have a storage unit (not illustrated) that stores the measurement result between the reception unit and the calculation unit 52. Furthermore, the calculation unit 52 may appropriately extract data from the storage unit and perform noise cut processing.

Examples of the analysis apparatus 50 include electronic equipment such as, for example, a smartphone, a tablet terminal, a smart watch, and a computer.

The system for managing a health condition of grown-up cattle 41 preferably includes input means for inputting an awakening time or a sleeping time. Thus, awakening information and sleeping information can be input from the input means.

The system for managing a health condition of grown-up cattle 41 preferably includes body temperature measuring means. When feed intake is insufficient, a heat production amount decreases, and a body temperature also decreases accordingly. In addition, in order to grasp a heat stress situation in a summer season, the fasting state can be managed in more detail by acquiring body temperature data by the body temperature measuring means.

The system for managing a health condition of grown-up cattle 41 may have a respiration rate measuring instrument as the measurement instrument 60. Symptoms of cough can also be detected by changes in respiration. Specifically, as means for measuring a respiration rate, the respiration rate can be measured by attaching, to the grown-up cattle, a displacement sensor that detects a change in length to a site where a circumferential length changes due to respiration. Examples of the displacement sensor include displacement sensors of a type in which a resistance is changed by displacement and a type in which a capacitance is changed.

Next, a system for managing a health condition of grown-up cattle 42 according to a second embodiment will be described with reference to FIG. 9. Components similar to those of the system for managing a health condition of grown-up cattle 41 according to the first embodiment are denoted by the same reference signs, and description thereof will be omitted.

As illustrated in FIG. 9, the system for managing a health condition of grown-up cattle 42 may have the measurement instrument 60, a web server 59, and a determination result notifier 62, and may have a transceiver 63. In addition, the web server 59 has the measurement unit 51, the calculation unit 52, the processing unit 53, and the determination unit 54. The determination result notifier 62 has the determination result notifying unit 55. Further, the measurement instrument 60 may have the beat interval measuring unit 51a and the acceleration measuring unit 51b. One example of a flow of the system for managing a health condition of grown-up cattle 42 will be described below.

The measurement instrument 60 is attached to the chest or the like of the grown-up cattle, and the beat interval measuring unit 51a and the acceleration measuring unit 51b of the measurement unit 51 measure the beat intervals and the accelerations. Subsequently, the measurement result is transmitted to the calculation unit 52 of the web server 59, and the calculation unit 52 calculates the negative accelerations in the body height direction of the grown-up cattle, and the beat value. The negative accelerations and the beat values may be calculated by the measurement unit 51. Subsequently, the processing unit 53 of the web server 59 obtains the regression formula of the negative accelerations in the body height direction and the beat values, and further obtains a beat value when a negative acceleration in the body height direction is 0 (at the time A₀) using the regression formula. Furthermore, these calculation results are sent to the determination unit 54 of the web server 59, and the determination unit 54 determines that the grown-up cattle is in the fasting state when the magnitude relationship of at least one of above formulae (1) to (5) is satisfied. These determination results are sent to the determination result notifying unit 55 of the determination result notifier 62, and are notified in a perceivable form of information indicating that the grown-up cattle is in the fasting state. The determination result notifying unit 55 performs, for example, display indicating that the grown-up cattle is in the fasting state, generation of a warning sound for notifying that the grown-up cattle is in the fasting state, and the like.

Note that at least part of data obtained by the calculation unit 52 and the processing unit 53 may be directly transmitted to the determination result notifying unit 55.

First, as another flow of the web server 59, similarly to the above flow, the beat intervals and the accelerations are measured, the measurement result is transmitted to the calculation unit 52 of the web server 59, and the calculation unit 52 calculates the negative accelerations in the body height direction of the grown-up cattle and the beat values. Furthermore, the calculation unit 52 calculates the side-lying time on the basis of the negative accelerations in the body height direction. The negative accelerations, the beat values, and the side-lying time may be calculated by the measurement unit 51. Subsequently, the processing unit 53 of the web server 59 obtains the regression formula of the negative accelerations in the body height direction and the beat values, and further obtains a beat value when a negative acceleration in the body height direction is 0 (at the time A₀) using the regression formula. These calculation results are sent to the determination unit 54 of the web server 59, and the determination unit 54 creates the two-dimensional coordinate system with the beat value at the time A₀ and the side-lying time as axes, fractionates the two-dimensional coordinate system into the four regions, and determines which one of the four regions the calculation results correspond to. The determination result is sent to the determination result notifying unit 55, and is notified in the perceivable form of information. The determination result notifying unit 55 performs, for example, the display, the generation of the warning sound, or the like indicating that the grown-up cattle is in the fasting state or in the insufficient side-lying state.

Examples of the transceiver 63 configuring a transition and reception unit 57 include a transceiver of a short-range wireless communication technology Bluetooth or the like. As a result, the transmission from the measurement unit 51 to the calculation unit 52 can be performed wirelessly. Note that the transmission may be performed by wire.

Examples of the web server 59 include a rental server and a cloud server, and the cloud server is preferable.

The transmission from the determination unit 54 to the determination result notifying unit 55 can be performed wirelessly. Note that the transmission may be performed by wire.

The web server 59 may have a reception unit (not illustrated) that receives the signal from the measurement unit 51, and may have, between the reception unit and the calculation unit 52, a storage unit (not illustrated) that stores the measurement result. Furthermore, the calculation unit 52 may appropriately extract data from the storage unit and perform noise cut processing.

Examples of the determination result notifier 62 configuring the determination result notifying unit 55 include electronic equipment such as a smartphone, a tablet terminal, a smartwatch, and a computer. Among them, the smartphone, the tablet terminal, and the smartwatch are preferable.

The system for managing a health condition of grown-up cattle 42 preferably includes input means for inputting the awakening time or the sleeping time, body temperature measuring means, a respiration rate measuring instrument, and the like. For details, reference may be made to the description of the system for managing a health condition of grown-up cattle 41.

A garment according to an embodiment includes at least a part of the system for managing a health condition of grown-up cattle. The garment for managing a health condition of grown-up cattle preferably has the measurement unit 51. The garment for managing a health condition of grown-up cattle may further have the calculation unit 52, the processing unit 53, and the determination unit 54, and may further have the determination result notifying unit 55. A form of the garment for managing a health condition of grown-up cattle is not particularly limited, and a belt-shaped article such as underwear or a belt is preferable. In addition, the garment preferably covers at least one site of the chest, the abdomen, the leg, the neck, and the head, more preferably covers the chest and/or the abdomen. Examples of the garment for managing a health condition of grown-up cattle include the biological information measuring garment 1 illustrated in FIG. 6. In addition, the garment preferably includes a detachable electronic unit.

An apparatus for managing a health condition of grown-up cattle according to an embodiment includes a measurement unit that measures beat intervals and accelerations in a predetermined period; a calculation unit that calculates at least one (hereinafter referred to as beat values) of average values of the beat intervals, variations in the beat intervals, beat numbers, HFs, or LF/HFs in the predetermined period; a processing unit that obtains a regression formula of the beat values and the accelerations, and obtains a beat value when an acceleration is 0 (hereinafter referred to as a time A₀) using the regression formula; and a determination unit that determines that the grown-up cattle is in a fasting state when a magnitude relationship of at least one of formulae (1) to (5) below is satisfied, and determines that the grown-up cattle is in a non-fasting state when none of magnitude relationships of formulae (1) to (5) below is satisfied.

the average value of beat intervals at the time A₀>A₁ (1)

the variation in beat intervals at the time A₀>A₂ (2)

the beat number at the time A₀<A₃ (3)

the HF at the time A₀>A₄ (4)

the LH/HF at the time A₀<A₅ (5)

The apparatus for managing a health condition of grown-up cattle according to the embodiment has the measurement unit, the calculation unit, a specification unit, and the determination unit. For details of units, the beat intervals, the accelerations, the beat value, the beat value at the time A₀, and formulae (1) to (5), the above description of the method for managing a health condition of grown-up cattle, the system for managing a health condition of grown-up cattle 41 according to the first embodiment, and the like can be referred to.

It is preferable that the determination unit determines that the state is a sufficient side-lying state when the side-lying time is a constant LI or more, and determines that the state is an insufficient side-lying state when the side-lying time is less than the constant L.

It is preferable that the determination unit creates a two-dimensional coordinate system with the beat value at the time A₀ and the side-lying time as axes and fractionates the two-dimensional coordinate system into four regions below:
Region 1: a region in which the beat value at the time A₀ does not satisfy any of the magnitude relationships of formulae (1) to (5) and the side-lying time is the constant L or more
Region 2: a region in which the beat value at the time A₀ does not satisfy any of the magnitude relationships of formulae (1) to (5) and the side-lying time is less than the constant L
Region 3: a region in which the beat value at the time A₀ satisfies at least one of the magnitude relationships of formulae (1) to (5) and the side-lying time is the constant L or more
Region 4: a region in which the beat value at the time A₀ satisfies at least one of the magnitude relationships of formulae (1) to (5) and the side-lying time is less than the constant L.

For these side-lying time, sufficient side-lying state, two-dimensional coordinate system, and the like, the above description of the method for managing a health condition of grown-up cattle, the system for managing a health condition of grown-up cattle 41 according to the first embodiment, and the like can be referred to.

Specific examples of the apparatus for managing a health condition of grow-up cattle include an apparatus having the measurement unit 51, the calculation unit 52, the processing unit 53, and the determination unit 54 of the system for managing a health condition of grown-up cattle 41. Furthermore, the apparatus for managing a health condition of grown-up cattle preferably has the determination result notifying unit 55. Furthermore, the apparatus for managing a health condition of grown-up cattle may have the transmission and reception unit 57. In addition, the apparatus for managing a health condition of grown-up cattle may be an apparatus including the measurement instrument 60 and the analysis apparatus 50.

The present application claims the benefit of priority based on Japanese Patent Application No. 2020-193644 filed on Nov. 20, 2020. The entire contents of the specification of Japanese Patent Application No. 2020-193644 filed on Nov. 20, 2020 are incorporated herein by reference.

### EXAMPLES

Hereinafter, the present invention will be more specifically described with reference to examples, but the present invention is not limited by examples below, and can be carried out with modifications within a range that can conform to the gist described above and below, and all of them are included in the technical scope of the present invention.

Biological Information Measuring Garment

Clothing fabric having a shape similar to that of the clothing fabric 10 illustrated in FIG. 6 was produced. First, a urethane sponge sheet manufactured by KAZUWA Corporation was cut into a substantially trapezoidal shape illustrated in first clothing fabric 10a as a base substrate. Specifically, the clothing fabric was cut into the substantially trapezoidal shape illustrated in the first clothing fabric 10a of FIG. 6 so as to have a maximum length of 33 cm in the front-rear direction, a minimum length of 12 cm in the front-rear direction, and a length of 93 cm in the body circumferential direction C. Furthermore, both surfaces were covered with a circular knitting fabric (ECO-1085) manufactured by Toray Industries, Inc. Next, a double lacquer mesh (FT6000) manufactured by Toray Industries, Inc. was cut into a trapezoidal shape illustrated in second clothing fabric 10b. Specifically, the clothing fabric was cut so as to have a lower side of 30 cm, an upper side of 9 cm, and a height of 45 cm. Thereafter, these were sewn by piping to form the main body portion 10c. Furthermore, urban twill manufactured by Masuda Co., Ltd. was cut into a size of 6 cm×140 cm, and a free magic tape having a size of 38 cm×130 cm and manufactured by Kuraray Fastening Co., Ltd. was attached to the main body portion 10c by sewing to form the first band portion 11. In addition, urban twill manufactured by Masuda Co., Ltd. was cut into a size of 6 cm×10 cm, and a metal buckle eight-shape ring attached thereto was fixed to the main body portion 10c by sewing to form the second band portion 12, thereby obtaining a garment for cattle. Electrodes were provided on the skin-side surface of the garment for the cattle thus obtained to produce a biological information measuring garment. Details are as described below.

As a resin, 20 parts by mass of nitrile rubber (Nipol DN003 manufactured by Zeon Corporation) was dissolved in 80 parts by mass of isophorone to prepare an NBR solution. To 100 parts by mass of the NBR solution, 110 parts of silver powder (“Agglomerated Silver Powder G-35” manufactured by Dowa Electronics Materials Co., Ltd., average powder diameter 5.9 μm) were mixed and kneaded with a 3 roll mill to obtain a stretchable silver paste.

Next, the stretchable silver paste was applied onto a release sheet and dried in a hot air drying oven at 120° C. for 30 minutes or more to prepare a sheet-shaped conductive layer with the release sheet.

Next, a polyurethane hot-melt sheet was bonded to a surface of the conductive layer of the sheet-shaped conductive layer with the release sheet, and then the release film was peeled off to obtain a sheet-shaped conductive layer with a polyurethane hot-melt sheet. The polyurethane hot-melt sheet was laminated using a hot press machine under a condition of a pressure of 0.5 kg/cm², a temperature of 130° C., and a pressing time of 20 seconds.

Next, a polyurethane hot-melt sheet side of the sheet-shaped conductive layer with a polyurethane hot-melt sheet having a length of 12 cm and a width of 2 cm was laminated on a polyurethane hot-melt sheet having a length of 13 cm and a width of 2.4 cm to produce a laminate of the polyurethane hot-melt sheet and the sheet-shaped conductive layer. The polyurethane hot-melt sheet corresponds to the first insulating layer described above.

Next, in a region of 5 cm in length and 2.4 cm in width, a same polyurethane hot-melt sheet as that of which the first insulating layer was formed was laminated from a portion separated from an end by 2 cm so as to cover the first insulating layer and a part of the conductive layer, thereby forming the second insulating layer on a part of the conductive layer. That is, a skin contact electrode was produced in which the electronic equipment connection portion having a length of 2 cm×a width of 2 cm with the conductive layer exposed at an end portion, an insulating portion having a laminated structure of the first insulating layer/the conductive layer/the second insulating layer, and an electrode having a length of 5 cm×a width of 2 cm with the conductive layer exposed at an opposite end portion were disposed in this order in a longitudinal direction.

Next, the two skin contact electrodes were attached to predetermined positions on the skin-side surface of the garment for cattle so as to be bilaterally symmetrical as illustrated in FIG. 6. Further, a snap hook as a connector was provided at each of the electronic equipment connection portions.

### Example 1

The biological information measuring garment 1 of FIG. 6 obtained as described above was worn on a Holstein (female, body weight: 550 kg, age: 26 months) as illustrated in FIG. 7, and a measurement instrument having functions of an accelerometer and an electrocardiograph was attached so that the accelerometer and the electrocardiograph are located in the chest. Further, as illustrated in FIG. 8, after the constant A₁ relating to the average value of the RRIs of formula (1): 1000 ms, the constant T relating to the negative acceleration in the body height direction: −0.7, and the constant L relating to the side-lying time: 12 were set as references in the analysis apparatus 50, the analysis apparatus 50 and the measurement instrument 60 were connected wirelessly. Thus, the system for managing a health condition of grown-up cattle 41 according to the first embodiment was configured. The beat intervals of the grown-up cattle in free stall breeding were measured for 1 hour between 16:00 and 17:00 by the measurement instrument 60. The accelerations were measured by the measurement instrument 60 from 16:00 to 24 hours. The measurement results showed that the average value of the beat intervals (RRIs) at the time A₀ was 1200 ms, and further, the side-lying time per 24 hours was 9 hours. That is, the magnitude relationship of formula (1) was satisfied, and the side-lying time was less than the constant L. Next, the determination result notifying unit 55 displayed the regions 1 to 4 of the two-dimensional coordinate system, and displayed that the measurement results and the determination result were located in the region 4. The region 4 is a region indicating that the grown-up cattle is in the fasting state and the side-lying time is insufficient, and the owner who looked at the display gave additional feed to the Holstein, and further performed replacement of a floor material and additional treatment. As the breeding was continued by this method, the milk production after the treatment increased by 10% with respect to the milk production before the treatment.

### Example 2

The biological information measuring garment 1 was worn on grown-up cattle similarly to example 1 described above except that a Holstein (female, body weight: 600 kg, age: 45 months) was used as a measurement target. Further, the analysis apparatus 50 set with the constant A4 relating to the average value of the HFs in formula (4): 1000, the constant T relating to the negative acceleration in the body height direction: −0.5, and the constant L relating to the side-lying time: 10 as references, and the measurement instrument 60 were wirelessly connected. Subsequently, the beat intervals were measured by the measurement instrument 60 for 2 hours between 9:00 and 11:00. The accelerations were measured by the measurement instrument 60 for 24 hours. The measurement results were that the HF at the time A₀ was 550, and further the side-lying time was 14 hours out of 24 hours. That is, the magnitude relationship of formula (4) was not satisfied, and the side-lying time was the constant L or more. Next, the determination result notifying unit 55 displayed the regions 1 to 4 of the two-dimensional coordinate system, and displayed that the measurement results and the determination result were located in the region 1. The region 1 is a region indicating that the grown-up cattle is in the non-fasting state and the side-lying time is sufficient, and the owner who had seen this display continuously bred the cattle in the same state without performing new treatment, and as a result, the owner was able to keep a predetermined milk quality.

### Example 3

The biological information measuring garment 1 was worn on grown-up cattle similarly to example 1 described above except that a Japanese black beef (female, body weight: 400 kg, age: 16 months) was used as a measurement target. Further, the constant A₂ relating to the average value of the RMSSDs in formula (2): 12, the constant T relating to the negative acceleration in the body height direction: −0.6, and the constant L relating to the side-lying time: 11 were set as references. The beat intervals of the grown-up cattle in grazing were measured for 3 hours from 13:00 to 16:00. The accelerations were measured for 24 hours. The measurement results were that the RMSSD at the time A₀ was 25 ms, and further the side-lying time was 14 hours out of 24 hours. That is, the magnitude relationship of formula (2) was satisfied, and the side-lying time was the constant L or more. As a result, the determination result notifying unit 55 displayed the regions 1 to 4 of the two-dimensional coordinate system, and displayed that the measurement results and the determination result were located in the region 3. The region 3 is a region indicating that the grown-up cattle is in the fasting state and the side-lying time is sufficient, and the owner who had seen this display added concentrated feed. Since the breeding was continued by this method, it was possible to spend time well without losing physical condition until pregnancy and delivery.

### DESCRIPTION OF REFERENCE SIGNS

1 biological information measuring garment
10 clothing fabric
10a first clothing fabric
10b second clothing fabric
10c main body portion
11 first band portion
12 second band portion
13a first tapered portion
13b second tapered portion
20 electrode
21 electronic equipment connection portion
31 first coupling member
32 second coupling member
40 grown-up cattle
C body circumferential direction
C1 one-end portion
C2 other-end portion
F front region
R central region
B rear region
41, 42 system for managing a health condition of grown-up cattle
50 analysis apparatus
51 measurement unit
51a beat interval measuring unit
51b acceleration measuring unit
52 calculation unit
53 processing unit
54 determination unit
55 determination result notifying unit
57 transmission and reception unit
59 web server
60 measurement instrument
62 determination result notifier
63 transceiver

1. A method for managing a health condition of grown-up cattle, comprising:
measuring beat intervals and accelerations in a predetermined period;
calculating at least one (hereinafter referred to as beat values) of average values of the beat intervals, variations in the beat intervals, beat numbers, HFs, or LF/HFs based on the beat intervals;
obtaining a regression formula of the beat values and the accelerations, and obtaining a beat value when an acceleration is 0 (hereinafter referred to as a time A₀) using the regression formula; and
determining that the grown-up cattle is in a fasting state when a magnitude relationship of at least one of following formulae (1) to (5) is satisfied, and determining that the grown-up cattle is in a non-fasting state when none of magnitude relationships of following formulae (1) to (5) is satisfied:
the average value of beat intervals at the time A₀>A₁ (1)
the variation in beat intervals at the time A₀>A₂ (2)
the beat number at the time A₀<A₃ (3)
the HF at the time A₀>A₄ (4)
the LH/HF at the time A₀<A₅ (5)
in the formulae (1) to (5), A₁ to A₅ are constants,
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

2. The method for managing a health condition of grown-up cattle according to claim 1, comprising setting, as negative acceleration, a value obtained by multiplying acceleration in a body height direction at a time of standing of the grown-up cattle by −1 when the acceleration in the body height direction is a positive value, setting a negative value as the negative acceleration when the acceleration is the negative value, and determining that the grown-up cattle is in a sufficient side-lying state when a time during which the negative acceleration is a constant T or more (hereinafter, referred to as a side-lying time) is a constant L or more, and determining that the grown-up cattle is in an insufficient side-lying state when the side-lying time is less than the constant L.

3. The method for managing a health condition of grown-up cattle according to claim 2, comprising creating a two-dimensional coordinate system with the beat value at the time A₀ and the side-lying time as axes, and fractionating the two-dimensional coordinate system into four regions below:
Region 1: a region in which the beat value at the time A₀ does not satisfy any of the magnitude relationships of the formulae (1) to (5) and the side-lying time is the constant L or more
Region 2: a region in which the beat value at the time A₀ does not satisfy any of the magnitude relationships of the formulae (1) to (5) and the side-lying time is less than the constant L
Region 3: a region in which the beat value at the time A₀ satisfies at least one of the magnitude relationships of the formulae (1) to (5) and the side-lying time is the constant L or more
Region 4: a region in which the beat value at the time A₀ satisfies at least one of the magnitude relationships of the formulae (1) to (5) and the side-lying time is less than the constant L.

4. The method for managing a health condition of grown-up cattle according to claim 1, wherein each of the average values of the beat intervals is an average value of RRIs each of which is an interval between an R wave and an R wave in an electrocardiographic signal.

5. The method for managing a health condition of grown-up cattle according to claim 1, wherein each of the beat numbers is a heart rate or a pulse rate.

6. The method for managing a health condition of grown-up cattle according to claim 1, wherein each of the variations in the beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

7. The method for managing a health condition of grown-up cattle according to claim 1, wherein the beat intervals are measured using a biological information measuring garment.

8. The method for managing a health condition of grown-up cattle according to claim 7, wherein
the biological information measuring garment includes:
a clothing fabric; and
an electrode provided on a skin-side surface side of the clothing fabric,
the clothing fabric includes a first band portion on one-end portion side in a body circumferential direction and a second band portion on an other-end portion side in the body circumferential direction, and
a front region ahead of a region from the first band portion to the second band portion of the clothing fabric has an area larger than a rear region behind the region from the first band portion to the second band portion of the clothing fabric.

9. A system for managing a health condition of grown-up cattle, comprising:
a measurement unit that measures beat intervals and accelerations in a predetermined period;
a calculation unit that calculates at least one (hereinafter referred to as beat values) of average values of the beat intervals, variations in the beat intervals, beat numbers, HFs, or LF/HFs based on the beat intervals;
a processing unit that obtains a regression formula of the beat values and the accelerations, and obtains a beat value when an acceleration is 0 (hereinafter referred to as a time A₀) using the regression formula; and
a determination unit that determines that the grown-up cattle is in a fasting state when a magnitude relationship of at least one of following formulae (1) to (5) is satisfied, and determines that the grown-up cattle is in a non-fasting state when none of magnitude relationships of following formulae (1) to (5) is satisfied:
the average value of beat intervals at the time A₀>A₁ (1)
the variation in beat intervals at the time A₀>A₂ (2)
the beat number at the time A₀<A₃ (3)
the HF at the time A₀>A₄ (4)
the LH/HF at the time A₀<A₅ (5)
in the formulae (1) to (5), A₁ to A₅ are constants,
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

10. The system for managing a health condition of grown-up cattle according to claim 9,
wherein the calculation unit sets, as negative acceleration, a value obtained by multiplying acceleration in a body height direction at a time of standing of the grown-up cattle by −1 when the acceleration in the body height direction is a positive value, sets a negative value as the negative acceleration when the acceleration is the negative value, and calculates a time during which the negative acceleration is a constant T or more (hereinafter, referred to as a side-lying time), and
the determination unit determines that the grown-up cattle is in a sufficient side-lying state when the side-lying time is a constant L or more, and determines that the grown-up cattle is in an insufficient side-lying state when the side-lying time is less than the constant L.

11. The system for managing a health condition of grown-up cattle according to claim 9, wherein the determination unit creates a two-dimensional coordinate system with the beat value at the time A₀ and the side-lying time as axes and fractionates the two-dimensional coordinate system into four regions below:
Region 1: a region in which the beat value at the time A₀ does not satisfy any of the magnitude relationships of the formulae (1) to (5) and the side-lying time is the constant L or more
Region 2: a region in which the beat value at the time A₀ does not satisfy any of the magnitude relationships of the formulae (1) to (5) and the side-lying time is less than the constant L
Region 3: a region in which the beat value at the time A₀ satisfies at least one of the magnitude relationships of the formulae (1) to (5) and the side-lying time is the constant L or more
Region 4: a region in which the beat value at the time A₀ satisfies at least one of the magnitude relationships of the formulae (1) to (5) and the side-lying time is less than the constant L.

12. The system for managing a health condition of grown-up cattle according to claim 9, wherein each of the average values of the beat intervals is an average value of RRIs each of which is an interval between an R wave and an R wave in an electrocardiographic signal.

13. The system for managing a health condition of grown-up cattle according to claim 9, wherein each of the beat numbers is a heart rate or a pulse rate.

14. The system for managing a health condition of grown-up cattle according to claim 9, wherein each of the variations in the beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

15. The system for managing a health condition of grown-up cattle according to claim 9,
wherein the measurement unit is provided on a clothing fabric of a biological information measuring garment,
the clothing fabric includes a first band portion on a one-end portion side in a body circumferential direction and a second band portion on an other-end portion side in the body circumferential direction, and
a front region ahead of a region from the first band portion to the second band portion of the clothing fabric has an area larger than a rear region behind the region from the first band portion to the second band portion of the clothing fabric.

16. A garment comprising at least a part of the system for managing a health condition of grown-up cattle according to claim 9.

17. An apparatus for managing a health condition of grown-up cattle, comprising:
a measurement unit that measures beat intervals and accelerations in a predetermined period;
a calculation unit that calculates at least one (hereinafter referred to as beat values) of average values of the beat intervals, variations in the beat intervals, beat numbers, HFs, or LF/HFs based on the beat intervals;
a processing unit that obtains a regression formula of the beat values and the accelerations, and obtains a beat value when an acceleration is 0 (hereinafter referred to as a time A₀) using the regression formula; and
a determination unit that determines that the grown-up cattle is in a fasting state when a magnitude relationship of at least one of following formulae (1) to (5) is satisfied, and determines that the grown-up cattle is in a non-fasting state when none of magnitude relationships of following formulae (1) to (5) is satisfied:
the average value of beat intervals at the time A₀>A₁ (1)
the variation in beat intervals at the time A₀>A₂ (2)
the beat number at the time A₀<A₃ (3)
the HF at the time A₀>A₄ (4)
the LH/HF at the time A₀<A₅ (5)
in the formulae (1) to (5), A₁ to A₅ are constants,
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.
